(19) United States
(12) Patent Application Publication
(10) Pub. No.: US 2012/0059254 A1
(43) Pub. Date: Mar. 8, 2012

(54) APPARATUS AND METHOD FOR PHASE-SPACE REDUCTION FOR IMAGING OF FLUORESCING, SCATTERING AND/OR ABSORBING STRUCTURES

(76) Inventors: Wang Lifan, College Station, TX (US); Carl R. Pennypacker, El Cerrito, CA (US); William Sheehan, Willmar, MN (US); James W. Gee, JR., Lake Geneva, WI (US); Michael Piontek, Chicago, IL (US)

(21) Appl. No.: 12/450,151

(22) PCT Filed: Mar. 14, 2008
(86) PCT No.: PCT/US08/34450

§ 371 Date: Nov. 16, 2011

Related U.S. Application Data
(60) Provisional application No. 60/918,006, filed on Mar. 14, 2007.

(51) Int. Cl. A61B 6/00; A61B 1/06; G01N 21/64; A61B 18/18; G01N 21/27; G01J 3/00

(52) U.S. Cl. 600/431; 356/73; 356/51; 250/459.1; 250/458.1; 606/2; 600/178; 600/476

(57) ABSTRACT

A method and apparatus are disclosed for utilizing light, including ultraviolet, optical and/or infrared, for detecting a body in an object, such as biomaterial or tissue, animal and/or human tissue. The body or object may be made fluorescent by the use of dyes or agent. Light is used to illuminate the body and object and the scattered light, fluorescent and/or emitted light, reflected light and transmitted light are detected and used to reconstruct the body and/or object using an iterative analysis. Further, the method and apparatus may be extended to endoscopic applications to make subcutaneous images of internal tissue above, on, in or beyond endoscopic pathways such as esophagus, stomach, colon, bronchial tubes and/or other openings, cavities and spaces animate or inanimate, and in man-made or industrial materials as carbon/resin structures.

### CROSS-REFERENCE TO RELATED APPLICATIONS

This is a United States Patent Cooperation Treaty (PCT) Patent Application which is a continuation-in-part of and claims priority and the filing dates of Provisional Application No. 60/918,006, filed Mar. 14, 2007, entitled “Apparatus and Method for Phase-Space Reduction for Measuring Sub-Surface Scattering and Absorption Centers”, in the names of inventors: Lifan Wang, Carl Pennypacker, William Sheehan, James W. Gee, Jr., and Michael Piontek and Provisional application Ser. No. ______, filed Mar. 4, 2008, entitled “Apparatus and Method for Phase-Space Reduction for Imaging of Fluorescing, Scattering and/or Absorbing Structures”, in the names of inventors: Lifan Wang, Carl Pennypacker, William Sheehan, James W. Gee, Jr., and Michael Piontek, both of which are herein incorporated by reference, and relate to a method and apparatus for phase-space reduction and measuring sub-surface scattering and absorption centers using ultraviolet, optical and/or infrared light, and in particular in human or other objects for imaging and measuring using fluorescing, scattering and/or absorption, and can be used endoscopically or externally.

### BACKGROUND OF THE INVENTION

It is well known to use light in various forms, such as x-ray etc., to construct an object, descriptional data, or image based on the detected, fluorescing, absorbed, transmitted and scattered light. For example see Block et al., U.S. Pat. No. 6,420,709, Marchitto, et al., U.S. Pat. No. 6,889,075, Van Der Mark, et al., U.S. Pat. No. 6,718,195, Flock, et al., Publication No. US/2001/0027273 (now U.S. Pat. No. 7,006,861), and Chan et al. (U.S. Pat. No. 6,175,759) all of which are herein incorporated by reference. Some of the prior art techniques used hazardous radiation as an illuminating source, while other techniques required complicated and expensive equipment to obtain and display or reconstruct an image.

### SUMMARY OF THE INVENTION

The present invention is for a method and apparatus using non-hazardous sources for illumination and techniques to utilize ultraviolet, optical and/or infrared light to obtain images of biological, plant, animal, human and certain inanimate objects, using reflected, scattered, absorbed, fluoresced, (usually, but not limited to, light excited by one wavelength of light and emitting another longer wavelength of light), and/or transmitted ultraviolet, optical, and/or infrared light to compute, construct and/or form or reconstruct the image desired. In more detail apparatus and methods are disclosed which, we maintain, can detect different objects, such as tumors, cancer, Traumatic Brain Injury (TBI), blood clots, blood flow, and other structures and functions of clinical interest subcutaneously and non-invasively, with depth penetration of at least and/or greater than approximately one centimeter, up to say four centimeters. Differentiation from other tissues is by the scattering, transmission, and absorption characteristics, parameters that are usually different for blood, lipids, body fluids, and other subcutaneous tissues and organs, or by emitted heat or fluoresced light.

Besides usefulness in biological materials, this device is useful in certain organic, inorganic, and non-biologic materials such as certain plastics, such as polymers, etc. This device can emit ultraviolet, infrared or optical light of various frequencies and polarizations into the subject. By analyzing the resultant reflectance, transmission, absorption, and scattering by the target and intervening or imbedding media, it is possible to solve for the underlying constituents and spatial distribution in the subject and locate the differentiated matter. This detector works at power levels and wavelengths that are harmless to animals or humans, even with prolonged exposure. Hence, some significant safety, ease of use and ubiquitous use of this apparatus/method ensues. For example, one could imagine such a device in a normal Family Practice Office, where pre-screening and treatment for breast cancer could occur at this point of care, or in a battlefield hospital to check for Traumatic Brain Injury (TBI).

A further claim is that by carefully tuning and illuminating a potential source of interest, the tumor (including cancerous ones) may be heated momentarily (sufficiently long to accomplish the result) to about 113 degrees Fahrenheit to kill the tumor, and surrounding tissues remain much cooler and undamaged. This temperature is well known, by a process called hyperthermia, to kill cancers ((see, e.g., http://www.cancer.govkancertopics/factsheet/therapy/hyperthermia) for data on hyperthermia studies).

This apparatus includes a new device (herein termed “collimator”, although this device is unique which allows this apparatus and method or system to function. The collimator can be in the form of a separate illuminating collimator and a detector or detecting collimator or combined into a single illumo-detector collimator. While it is preferable to have a collimator at the upstream (with respect to photon travel) distal end (entry place) in some situations the collimator may be located further downstream or even dispensed with. Such alternatives may have somewhat degraded but yet useable performance, than when the detecting collimator is located at the distal end.

The methods asserted would be useful in solving underlying radiactive transfer problem of light through a confused medium, using polarization, frequency, collimation, and other possible constraints. The “Phase Space” of the illuminating source is key, phase space being defined as the entry point and the velocity unit vector of incident photons. An important component of our device are the collimators, which in various forms are described below.

The technique is designed for use with transmitted light, absorbed light, scattered or reflected light, or some combination thereof. It also applies to a wide variety of geometries between the illumination source and the detectors. Environmental background light can reduced by shielding.

In essence, the technique is as follows. It is well established that, for example, human bone, organs, and soft tissues are at least somewhat transparent in appropriate ultraviolet, optical and infrared frequencies (viz., certain frequencies of light can penetrate the constituents of the human body, with some efficiency). Relative to reference tissues, malignant tissues, tissues without vascularization (such as brain trauma-Traumatic Brain Injury) and others of special clinical significance have different but characteristic scattering, fluorescing (in the presence or absence of fluoresing agents) and absorption functions. Prior art has claimed methods of using different frequencies (Gee and Pennypacker U.S. Pat. No. 7,158,660, which is incorporated herein by reference, Marchitto, et al, U.S. Pat. No. 6,889,075, issued May 3, 2005), different polarizations (Flock, Stephen T. et al, Publication No. US/2001/0027273, published Oct. 4, 2001, (now U.S. Pat. No. 7,006,861)), and other scattering characteristics.

This application asserts that measures with good detail and signal-to-noise ratios of the three-dimensional scattered pattern of light, together with data indicating scattering as a function of polarization, photon direction, fluorescing and frequency, allow a unique and restrictive reconstruction of the spatial location of scattering centers and, absorption and/or fluorescing features which are non-homogeneous to the embedding tissues. For the present invention other health-related targets are of interest, such as endoscopic internal applications, as are industrial fabrication and testing, such as discovering fracture zones or weaknesses or any strength-related compromises in, for example, carbon-epoxy or other resins or other structures. The present invention also as noted above relates to the utilization of fluorescence and also extends the invention with or without fluorescence to internal endoscopic applications. Differentiation of scattering, absorbing, emitting and/or fluorescing objects by a number of measured variables is used, included spectral distribution of all forms of the light signal, polarization, spatial dependence, and other characteristics of the input emerging and radiation.

With respect to inanimate subjects, including humans, one would inject in differentiating, say absorbing material, which has properties to distinguish the target from its environs (say surrounding tissue), providing the material has no harmful effects, to help establish an image. For example, ICN-Green will absorb light at certain frequencies and fluoresce at a different frequency could be used to delineate the target from the environs, or vice versa, (depending upon whether the material, ICN-Green or other, is located in the target or environs). This phenomenon would be useful in situations with or without detection of any subsequent emission post exitation. With respect to inanimate matters, the range of materials that could be used is broader as there is less or little concern with damage to the subject being studied. That does not mean no concern whatsoever. When doing nondestructive testing, for example, in a carbon-resin structure for an airframe, where the airframe is to be subsequently utilized if it passes, no use would be made of any material which would attack the subject, the carbon fiber, the laminate, the resin and/or bond. If the testing is of a destructive nature, then there would be less concern in the selection and use of a differentiating material.

As noted the method and apparatus of the present invention illuminating and detection with or without fluorescing, can be provided in external and/or internal or endoscopic applications for animate or inanimate subjects. Besides usefulness in biological materials, this device is useful in certain organic, inorganic, and non-biologic materials such as certain laminates and plastics, such as polymers, etc. The device of the invention may emit ultraviolet, infrared or optical light of various frequencies and polarizations into the subject. By analyzing the resultant reflectance, fluorescent or other emission, transmission, absorption, and scattering by the target and intervening or imbedding media, it is possible to solve for the underlying constituents and spatial distribution in the subject media and locate the differentiated matter. The fluorescing substance could be injected directly or indirectly or otherwise placed into a structure of interest or the patient, which could be a vascular structure, or activity or lack of activity, or presence or absence of fluorescing material. For example, another form of providing the fluorescing material would be to take the same orally (which could be considered to be another form of injection).

For example, Traumatic Brain Injury (TBI) manifests itself with less blood flow in areas of the brain injured by some external (usually) agent, such as explosive projectiles or shock waves or other explosive debris, a rock or pipe, or an auto or sports-related accident. Patients would exhibit a deficit of the usually injected blood carried fluorescing agent or lack of blood flow, using methods described below. That is, areas around the wound or trauma would show evidence of the transport of the fluorescing agent, whereas the injured area would show less or no blood transport to this region. In addition, using the spectral and polarization information present and differentiating oxy- and deoxy-hemoglobin, flesh, bone, and other animate and inanimate structures, allows one to understand the structure of the underlying surface. This detector works at power levels and wavelengths that are harmless to humans, even with prolonged exposure (approximately 1 watt power spread over a few sq. centimeters in one embodiment). Higher power levels could be used with industrial or inanimate materials, resulting in deeper penetration and more detailed elucidation of the underlying structure. The present invention in various applications may provide images say of a depth of from or on the surface to 1 cm to as far as 4 cm below or beyond the surface, including in endoscopic applications used heretofore or in the future to detect such matters and develop images. As the apparatus and method used even with the fluorescent and/or endoscopic forms is inexpensive compared to say, a CAT scan device, it makes such screening or other uses possible in local hospitals, clinics, third world countries, even rural areas, airports, public arenas, sports events, doctors' offices, emergency rooms, ambulances, and trauma care centers. As an image acquired with wavelengths that are not fluorescing can be subtracted from the image with the fluorescing area of interest, which could include the target or the area around the target, a very high signal-to-noise ratio image can be acquired, with very little background interference. With such approach only the areas of interest are highlighted in the image acquired by subtraction of the two (or more) images. Such approach would reduce or eliminate noise and interference from matters such as hair, bone, skull and/or other non-vascular structures in, for example, a TBI imaging.

A further advantage as noted above and in our earliest provisional application is for example, a tumor (including cancerous ones) may be heated momentarily to kill the tumor, and surrounding tissues remain much cooler and undamaged. With a fluorescing agent and/or use of selected wavelengths of light as noted in our later provisional application could expedite preferential absorption of energy in the tumor or the surrounding areas, which have higher vascularization. Thus, one could absorb preferentially energy in the area of interest with such a system, by sending in light that absorbs much more preferentially than the surrounding flesh, hence depositing energy in the tumor much more efficiently, with no danger to the patient.

This apparatus may include a device, herein termed illumo-detector which, as noted can be a separate illuminator and a separate detector or a combination unit carrying out both functions. The technique is designed for use with transmitted light, absorbed light, emanated light, fluorescing light, scattered light and/or reflected light, or some combination thereof. It also applies to a wide variety of geometries between the illumination source and the detector.

### BRIEF DESCRIPTION OF THE DRAWINGS

FIG. 1 is a simple schematic diagram of a first embodiment apparatus of and for performing the method of the present invention.

FIG. 1A is a schematic of a laser or light, two mirror alignment checking apparatus.

FIG. 2 is a schematic of light scattering as a function of polarization.

FIG. 3 is a schematic of an input (illuminator) and detector portion of the apparatus of and for practicing the method of the present invention.

FIG. 3A is a perspective schematic of a collimator tube (for the illuminator and/or detector) made of several sections of optical glass or fiber that has an absorbent or black coating on its outside surface and ends, with light transmitting collinear aligning small openings or pinholes in each end of the segments that can be stacked several in a tube.

FIG. 4 is further schematic of the input or illuminating portion of the apparatus of and a method of the present invention.

FIG. 5 is a schematic of another embodiment of an apparatus of and method for practicing the present invention, utilizing primarily reflected light suited for Traumatic Brain Injury.

FIG. 6 is a schematic of yet another embodiment of apparatus of and method for practicing the present invention.

FIG. 6A is a table of dimensions of the components of the present invention.

FIG. 7 is a schematic of the collimator device of the present invention.

FIG. 8 is a schematic of a light source using a micro mirror array (mma) to control input of light into the collimator.

FIG. 9 is a schematic side view of another embodiment of light scattering after penetrating the skull, for example, in a TBI application, and exciting a target injected with a fluorescing dye or agent.

FIG. 10 is a schematic of an input and detector portion of the apparatus and method for practicing the present invention utilizing an illumo-detector strip in place on a patient's head.

FIG. 11 is a schematic of another embodiment of apparatus of and method for practicing the present invention without the strip of FIG. 10.

FIG. 12 is a graph showing the excitation and emission response for a typical fluorescing dye.

FIG. 13 is a schematic of the application of the present invention in an endoscopic device.

FIG. 14 is a schematic of the present invention in the form of an internal endoscope.

FIG. 15 is a schematic diagram illustrating how using normal body pathways (e.g., colon, intestine, trachea, bronchial tubes, esophagi, open body space, etc.) the present invention in endoscopic form may detect anomalies on, in and/or up to 4 cm away from the surface of the pathway.

### DETAILED DESCRIPTION OF THE INVENTION

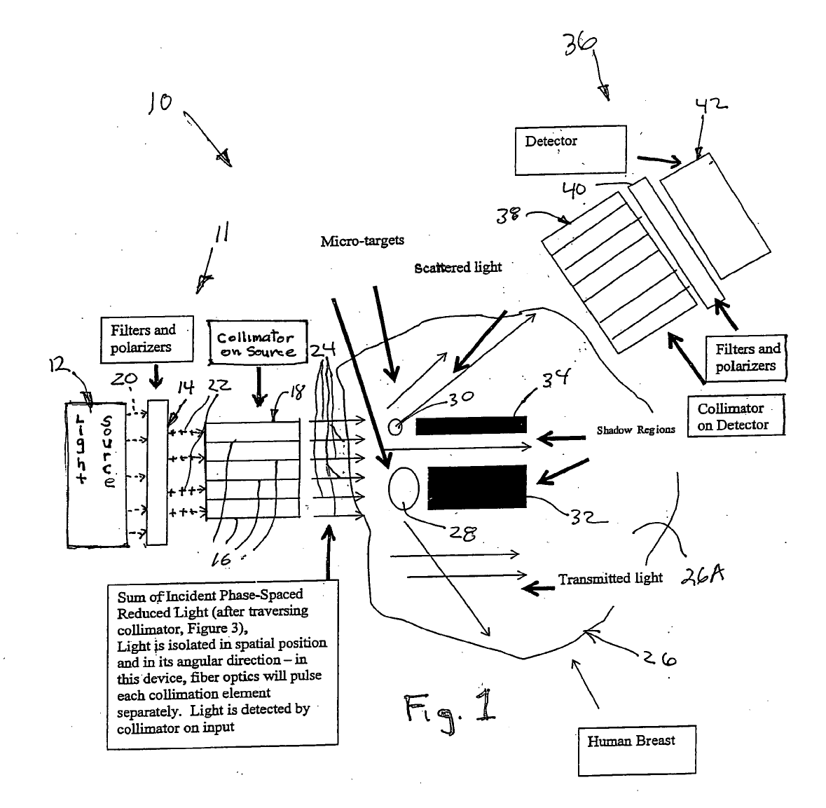

Referring to FIG. 1, a first embodiment of apparatus 10 of and for practicing the present invention is shown. Starting from the left, it includes or comprises an input or illuminator unit 11, light source 12, optionally filters and/or polarizers 14, preferably, and a plurality of collimator tubes 16 forming an input collimator 18. The light source could be photo diodes, diode lasers, or incandescent or other lighting, with or without filters, with the purpose of injecting light into the subject with phase-space reduced beams. As shown by the dotted arrows 20, light leaves the light source 12 and enters and is altered in the filters/polarizers 14. Light from the filters/polarizers 14, if used, or from the light source 12, if filters/polarizers are not used, then enters collimator 18 and collimator tubes 16, as indicated by the plus arrows 22. From there collimated or collinear light 24 strikes the target 26, in this instances a human breast.

The collinear light 24, is to some degree reflected, scattered, absorbed and transmitted through the target 26. As noted within, the target 26 could be two internal object targets, a large one 28 and a smaller one 30. The collinear light will cause or create shadowed areas (not illuminated) 32 and 34, with the shadows' cross sections corresponding to the cross sections of the targets 28 and 30.

In order to collect the scattered, reflected and transmitted light, if any, a detector portion 36 is provided, and can comprise an output collimator 38 similar to collimator 18, and output filters and/or polarizers 40, similar to filters/polarizers 12 and a detector unit 42. Light from the target enters the output collimator 38, and if used the filters/polarizers 40, and then the detector unit 42.

The fundamental science is schematically indicated in FIG. 1. From the above it is shown that light from the light source 12 is sent through the input collimator 18, which is similar to the detection or output collimator 38, which may be transformed by filters and/or polarizers 14 to collimated and/or collinear light 24. The collimated light 24 is incident and then propagates through the human breast or target 26. Light—say, laser light—traveling unscattered and collinearly is called or termed “ballistic photons” in some literature and prior art. The detector 36 would be located to gather the reflected and scattered collinear light to determine the light absorbed which help characterize the object. For example the input light source unit 11 and/or the output detectors 36 could be moved relatively radically about the target 26 and/or large and small targets 28 and 30. This apparatus 10 could provide “shadow images” 32 or 34 of the targets 28 or 30. More information about the basic principles is to be found in Alfano, et al, who, unlike in the present invention, try to eliminate or reduce the effect of scattered light by employing a time gate. The time gate concept is difficult if not impossible to carry out. Whereas in the Alfano, et al. prior art an attempt is made to reduce or eliminate the effect of scattered light, in the present invention scattered light is actually utilized and considered in obtaining a solution and analyzed to obtain and form the resultant image.

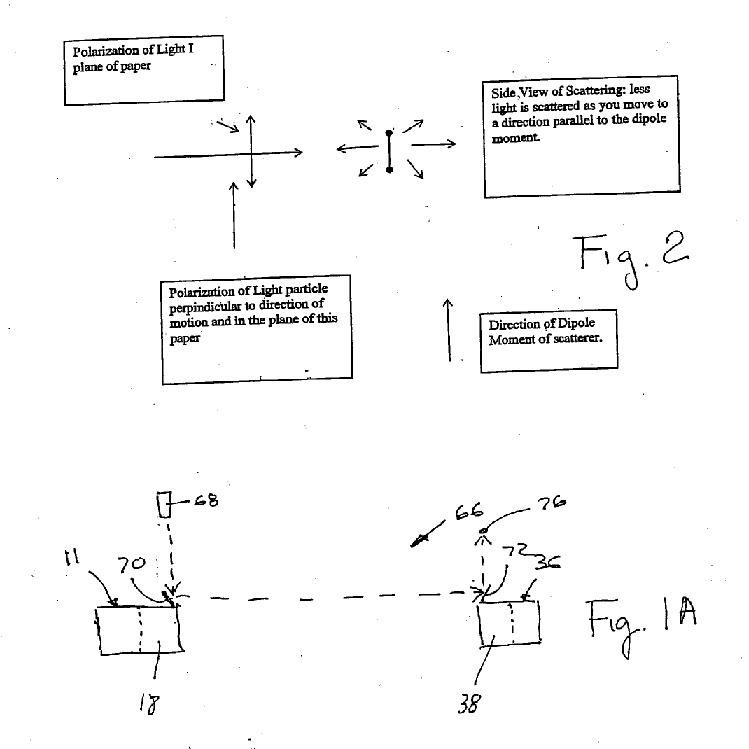

FIG. 2 shows how the polarized collinear light 24 scatters.

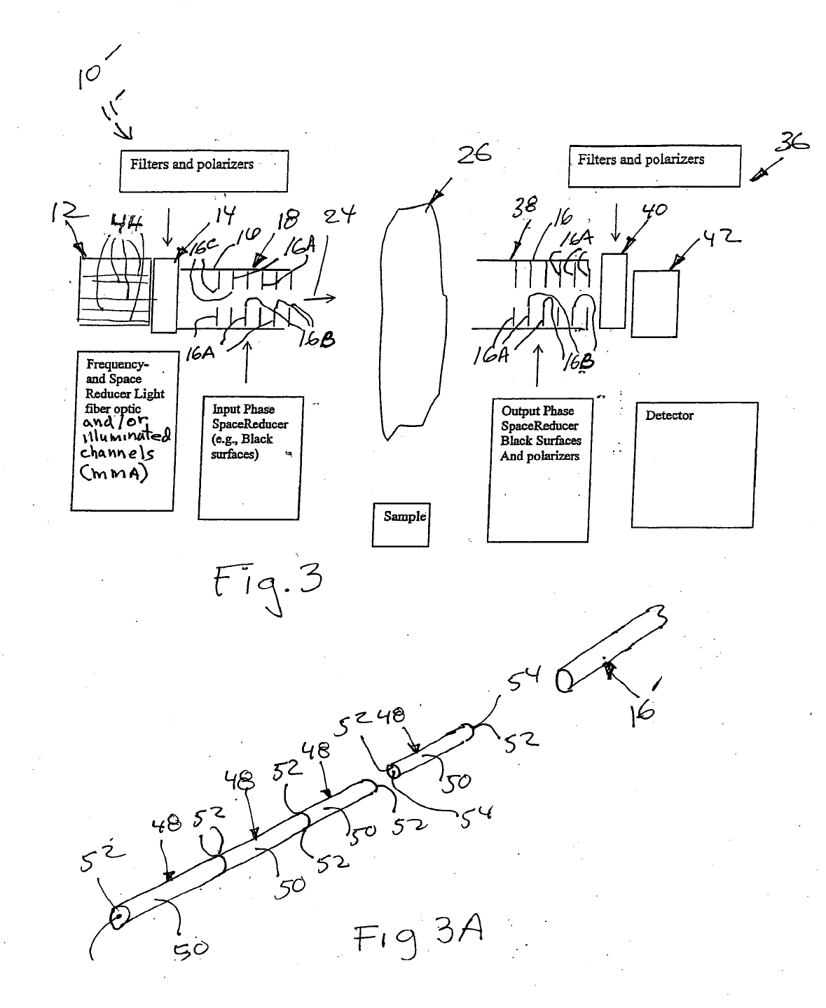

FIG. 3 shows another embodiment 10′ generally similar to that shown in FIG. 1 in an aligned position of the input unit 11′, target 26, and detector unit 36. To the extent it is the same, the same reference numerals are used. To the extent, if any, it differs, different reference numerals are provided.

Referring to FIG. 3, in one potential embodiment of our apparatus 10′ of the present invention, light 24 of a given polarization (see FIG. 2) is channeled into the subject 26, by pulsing individual optic fibers 44, which then are injected into a collimator 18 (see FIG. 3). As shown in FIG. 3A, the collimator 18 (and 38) could be constructed of plurality fiber optic or glass rods segments 48 with non-reflecting (absorbing or black) outer surfaces (cylindrical surface 50 and ends 52) formed as by coating thereon. The ends 52 have small openings or pin holes (say of 0.01 mm to 1 mm diameter or approximately 0.00008 mm² to 0.8 mm² area) in both ends of each segment 50, with a plurality or several segments, say 5-10, stacked to form the collimator. As a compromise giving good photon discrimination and ease of forming, manufacturing and aligning an opening of about 0.1 mm in diameter or length and width might be suitable, which is an area of about 0.01 mm² to 0.008 mm², depending on whether of a square or round cross-section. The rod segments 48 can be of any cross section but are preferably round or square and have ends cut and polished at right angles. The pin holes 54 are generally in the center and collinear so as to pass light from one segment 48 to the next so that light can travel from the source to the target and is collinear when it hits the target or received by the detectors. For convenience and alignment, the various segments 48 may be placed in a collinear tube 16′.

Further a micro mirror array could also be used and would comprise a means for illuminating one or several selected collimator tubes 16 or 16′ at a time with the other tubes dark. This construction reduces the phase space of the input beam 24—that is, the beam enters the subject 26 or 26′ with small angular scatter and known (e.g., Cartesian coordinates x,y,z polar coordinates R,O, Phi) entry location on the subject. Then, the output light and including scattered, reflected and transmitted light, is measured by the detector across as many angles as necessary to attain adequate detection, say to capture 80% or more of the total scattered light or of sufficient data to attain adequate detection. Light from off the target is again made collinear and optionally filtered and polarized and received at the detector 42. Thus, this detected light will depend on the direction of the incoming light and the polarization of the beam. For example, FIG. 2 shows the simple case of a dipole scattering, where the length of the arrows indicate the scattering of light. No light is scattered in a direction parallel to the “dipole moment represented by the black dots”. We argue that illuminating the target with a second, but different polarized light, and then subtracting the two polarizations yields an informative map of scattered light. This scattered light can then be subtracted from the original image. The result defines in greater clarity details of the object of interest.

In FIG. 2 is a schematic of the behavior of light scattering as a function of polarization for an exemplary case: light polarized in the plane of the paper and perpendicular to the direction of motion of the photon. Light is scattered preferentially in angles not lying in the direction of the dipole moment, in the simple case proportional to the sin² of the angle between the dipole moment beam and the observer.

A co-alignment mechanism 66 (shown in FIG. 1A) to align the input collimator and output or detector collimator, say comprising a small laser 68 with mirrors connected to the body of the input and detecting collimators 11 or 18 and 36 or 38 could be used to maintain and measure the alignment of the input and output collimators, say by reflecting the laser beam onto an aligning target 76. If the light is off the target 76, the system 11 or 18 or 36 or 38 are out of alignment. Good alignment is necessary to be sure the diminution of the light at the detector is due to scattering by the main target 26 or 26′ or objects therein and not misalignment.

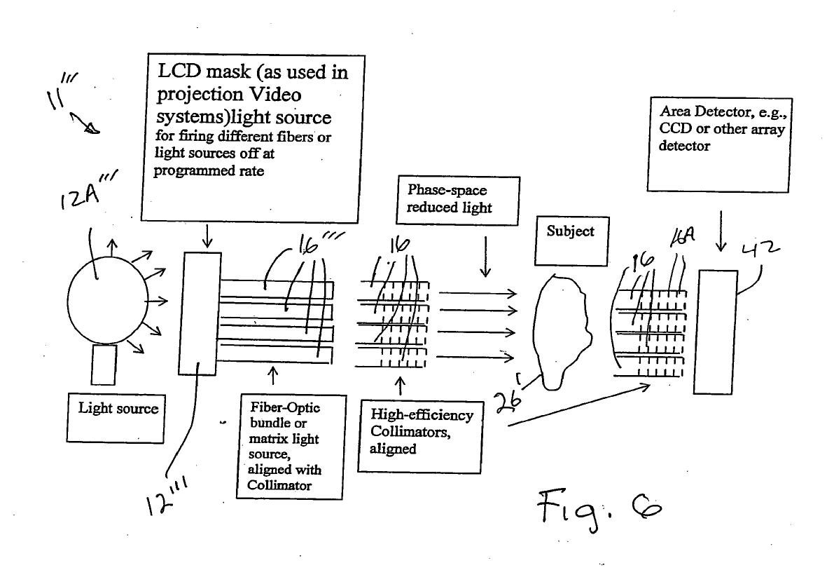

In these embodiments of our system, light of slightly different frequencies (for example 850 nm (nanometer) and, 750 nm, and also FIG. 6A) is emitted into the target. Since light scatters proportionately to frequency and differently for objects off beam, different images result, by a process of subtraction, corresponding to a range of frequencies (typically, but not limited to the case of low frequencies scattering less strongly than high frequencies). Furthermore, higher orders of scattering and transmission can be analyzed in order to understand the respective contributions of scattering and absorption in the target. (It is asserted objects off beam will exhibit a different frequency scattering response than those directly in the beam shadow.) It follows that an iterative solution can be found where the frequency dependence of the scattering of the absorbing component can be understood and compensated, thus achieving a clearer and more detailed image.

In yet a third use of these embodiments of our system, unpolarized light is channeled into the target, then light is measured with polarization sensitive detectors at many locations around the subject, say to capture 40% to 100%, and preferably 80% or more of the total scattered light or of sufficient data to attain adequate detection. We maintain that light of some polarizations will be more highly scattered. Polarizations can be changed by rotating polarizers, or swapping in different filters, and done uniformly across the collimator. This behavior allows the underlying target structure to be elucidated.

In a fourth use of these embodiments, after a cancer or unhealthy object is discovered, ballistic photons from the same, or more likely a different, phase space reducer mechanism 10 and 11 can be turned on, and the unhealthy object preferentially absorbs light, and is heated to about 113 degrees Fahrenheit or somewhat higher, within a range of plus 5 degrees Fahrenheit, which kills the cancer cells (“Hyperthermia”). Tissues around the cancer do not receive or absorb as much energy, and reach lower temperatures (under 113 degrees Fahrenheit), and hence will not be damaged. It is believed that this cellular altering heating could be accomplished with a power source (light) of 25 watt output or less. Preferably, the plurality of illuminating sources will be dispersed about the target so that the target or unhealthy object can be brought to the necessary temperature.

The phases of light incident on the illumo-detector could be constructed, after some model of the subject is constructed, to cancel out so some degree scattering and reflection off of material in the beam, as the heating beam moves to the tumor. This is through the well-known methods of adaptive optics (see e.g., http://en.wikipedia.org/wiki/Adaptive/optics.com). The phases may be adjusted by a deformable mirror, such as shown in FIG. 1 in the above internet reference.

A fifth use of these embodiments would allow the object to heat up by preferential absorption of light, and then the object could be discerned by the well-known method of thermal imaging, which can acquire different images at different wave lengths, say, 10 micron and 5 microns.

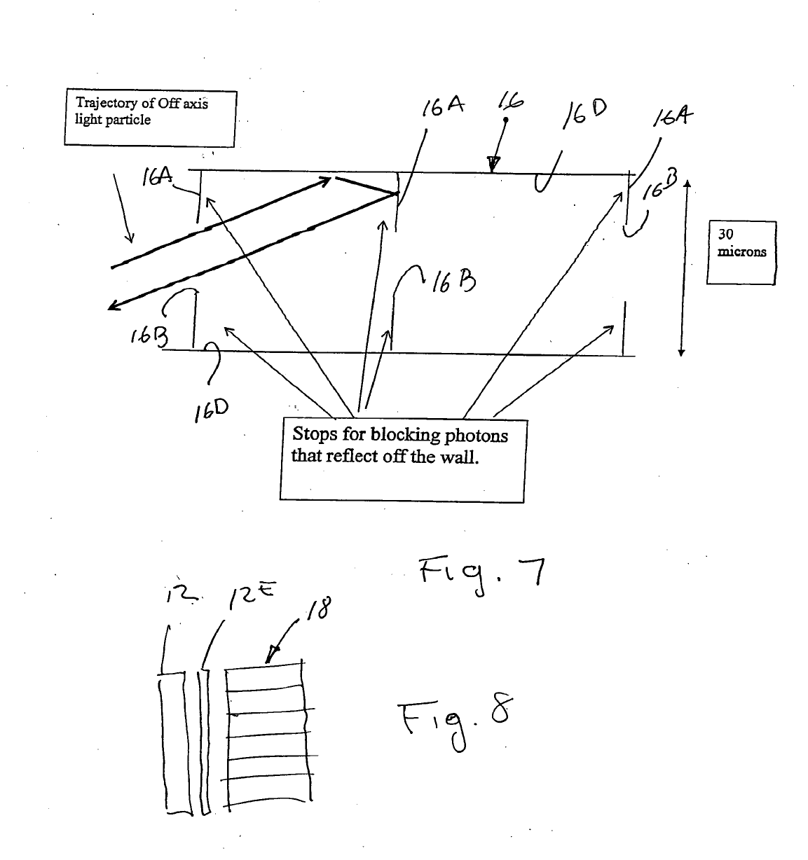

We assert that our device consists of a (probably movable) two-dimensional focal plane of detectors 36 and 42, with sensitivity ranging from ultraviolet or optical frequencies to the infrared. A light source 11 and 12, 14 in the collimation system, with the ability to change polarization as noted above and frequency as, for example, changed by filtering out (at 14 or 40) components from a general light spectrum, is the preferred light source, since such a source, when coupled to the detector and knowledge of the wavelengths and polarization, can help elucidate the scattering, transmission, and absorption properties of the underlying materials. A laser or other light source (at 12) can feed a micro mirror array (FIG. 8) to feed each “tube” 16 independently.

Data is collected for a number of incident angles between the laser (11, 12) and the target 26 so as to define the three-dimensional configuration of the target. Polarization and frequency dependences are used, in order to further elucidate the structure and exact position of the underlying scattering centers (objects in the subject target (such as 28 or 30 in 26).

Alfano, et al, have reported (see Technology in Cancer Research & Treatment, ISSN 1533-0346, Volume 4, Number 5, October (2005), ©Adenine Press (2005)) that time and frequency-gated laser light can be used to produce the images of shadows, e.g., of tumors, on the incident beam. However, it is very difficult to use only data obtained from light emitted before scattering effects the detector. Any time or frequency gate fast enough and stable enough is difficult to make and operate and severely limits the data available. We maintain that the method disclosed here represents a significant advance upon this prior art time and frequency gate method. Specifically, the exquisite selection of non-scattered ballistic photons, and the ability to select out singly-scattered or polarization selected photons give us more sensitivity and spatial resolution than Alfano et al. Timing, which has serious drawbacks, is no longer used; instead the total three-dimensional distribution of the scattering centers is worked out on the basis of frequency and polarization data. This permits exploitation of most or all available information present in the absorbed and scattered light.

A further advantage of this method and apparatus of the present invention allows just one tube 16 of the input collimator 18 to be illuminated at a time, and hence the signal detected at the co-aligned tube on the output collimator 38 next to the two-dimensional detector 42, with explicit knowledge of the input. This means that scattered light from other tubes 16 or positions in, for example, the breast are non-existent or greatly reduced. Hence, the signal can be seen against a much smaller background, than in the case when all of the background of the whole input collimator were illuminated simultaneously. Although multiply scattered photons have a small chance of scattering into the output (detector) collimator, most likely they will not have the same direction if they are incident on the co-aligned tube of the output collimator.

In addition—and this is important for imaging in the infrared, which contains some important biological windows for transmission by water, a component of most biological material, such as tissues and bone—the target subjects are usually sources of thermal emission which may at certain wavelength regions dominate the photons collected by the detector. The image subtraction technique we are proposing can efficiently remove this component, because scattered light is usually polarized whereas the thermal component is not. This concept is helpful in our system. Alternatively, we can also do the opposite—infrared light can be preferentially absorbed by tissues, which then heats them up, and causes them to emit more thermal photons. Then, not observing or subtracting polarized components allows one to see sources of thermal emission in the subject. The polarization components can be from the input light, from the scattered light, the filter on the detector, or any combinations of one or more of these.

Principle of Operation of Preferred Embodiments, for example, infrared or optical light illuminates a portion of the human subject, e.g., a lobe of the brain or a mammary gland.

### List of Components of Preferred Embodiment:

1) Multiple two-dimensional imaging units, or ability to position one two-dimensional infrared imaging unit 36 around subject 28 and 26 and viewing from multiple angles in sequence. Such apparatus 10 could include collimators, such 38 as positioned in front of the detector, say 42. Multiply angles viewed should be such that collect approximately 80% or more of the scattered light.
2) Collimated light source, say 11, which reduces the phase space of the input beam (20 to 22 to 24). Individual tubes (16 of 18) can be turned on and off under control, if necessary. Alternatively, the whole array of input tubes 16 on the collimator 18 can be turned on and off simultaneously, for example, in synchrony with the human arterial pulse or other body function to establish a reference frame for an image not affected by the arterial pulse or other body function.
3) Collimated Light Detector 36 and 42, with filters and polarization components 40 which allows phase space reduced light to be detected, hence greatly increase signal to noise for detecting the target (28 or 30 in 26), for example, cancer, etc.
   4) A data analysis algorithmic scheme (to be developed) that allows recovery of the structure of the underlying scattering and absorption centers below the surface.
   5) Software for image reduction and analysis (to be developed), which can reduce the algorithm and data to produce bona-fide three-dimensional maps of heterogeneous tissue structures.

While yet to be developed for this invention, such steps 4) and 5) would be similar to data analysis and image reduction already accomplished for galaxy image obtained with the Hubbell telescope, or such as with atmospheric corrections, for large earth optical telescopes. Thus, there is considerable degree of certainty of accomplishing steps 4) and 5) above, considering the inventors associated with this invention: a medical doctor, brain imaging specialist, affiliated with the medical school of a major university, and consulting with the Veterans Administration Hospital on brain trauma, a PhD research physicist connected with a major university and its space science laboratory, having 30 years experience in designing imaging systems, data analysis, and finding/separating small signals from background noise, a PhD on the faculty with a major university with a background in radiactive transfer in super nova atmosphere and super nova polarimetry, and the director of space and atmospheric research engineering at a major university and its telescopic observatory.

Referring back to FIG. 1, we illustrate as follows. Consider the object 26 being scrutinized—say a mammary gland—as comprised of two volumetrically small micro-targets 28 and 30. Each micro-target 28 and 30 scatters, transmits, and absorbs the light, with some efficiency. The output at any point in space becomes the sum of scattered, and transmitted light from the micro-targets' combined effect on the beam, where light passing through one micro-target is in turn subjected to scattering, absorption, and transmission by the next micro target. In reality, the subject is composed of a plurality of micro-targets of various scales, dimensions, and depth. In addition, if the target absorbs enough energy from the beam, it may heat up and preferentially emit more thermal radiation than the surrounding tissue 26A of target 26.

For point scattering micro-targets, the physics is very clear and straightforward. We take the case of a point source with scattering coefficient S, absorption coefficient A, and transmission coefficient T. By illuminating the target from various angles, we can solve for these and derive the underlying spatial characteristics. In this way we deduce the structure of this (trivial) zero-dimensional target. Using our two-dimensional focal plane detector 42, we detect single-scattered photons from the object which contain information about properties of the material beyond the simple absorption features. The general, three-dimensional solution of the entire scattered radiation is the basis of our first provisional patent application.

A slightly more complex case involves two point objects of different material. The point objects are characterized by the coefficients (as above) of S, A, and T, and s, a, and t, respectively, situated next to each other. If they are illuminated, and S, A, T and s, a, t of each material are known, if in addition we are able to place some constraints on the geometry, then it is possible to solve for the underlying spatial distribution of the tissue in question. By incorporating more and more detectors and viewing angles, we achieve a higher resolution to smaller targets—say about one millimeter. The principles are illustrated in FIG. 1, showing schematically the preferred embodiment. The coefficients of scattering, absorption, and transmission of targets and subjects are mostly uniform within small variability among all humans and most animals. If fluorescence is involved, it is addressed further on in this application.

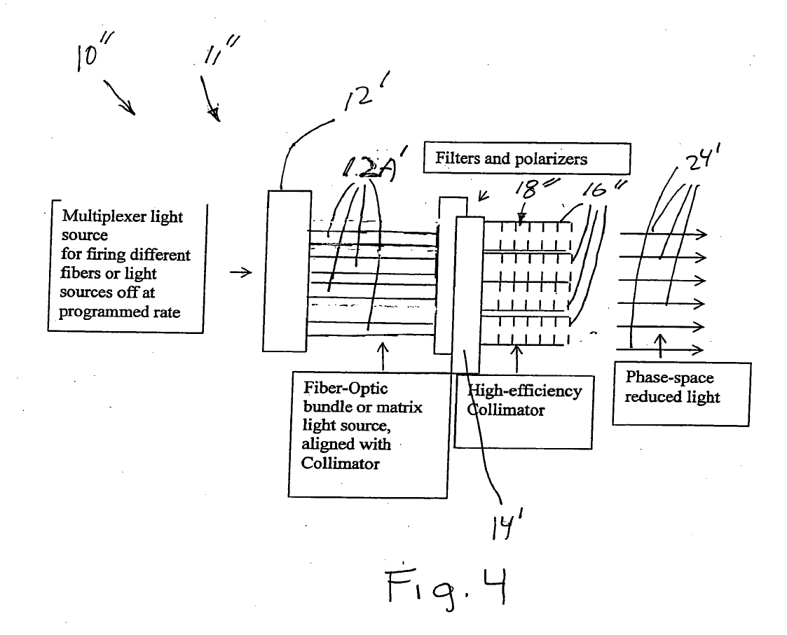

The following diagrams, FIGS. 3 and 4, illustrate details of the working system 10. FIG. 3 illustrates the make-up of one element or tube 16 of the Space Reduction System 10′. One would imagine a whole two-dimensional array of such units, stacked together to form a collimator system like shown in FIG. 8. The phase space reduction system could be made as mentioned on pages 11 and 12 and FIG. 3A herein, or made by casting, for example, a molten material around a mold, then melting the mold out later to form the tube 16 with internal absorbing, black baffles 16A with openings 16B therein. Note baffles 16A function similarly to the absorbing or black coatings applied to the outer surface ends 52, while the openings 16B function similarly to the pinholes or small openings 54 of the fiber optic rod version.

FIG. 4 shows a two-dimensional slice through a multi-element phase-reduction system 10″ with an input 11″, a multiplexer light source 12′ with fibers optic bundles 12A′ (for simplicity only four being shown—but these could be many more), filters/polarizers 14′ and collimator 18″ with (four) corresponding tubes 16″, that produce collinear, phase space reduced light 24′. Each fiber 12A′ or matrixed light source 12′ of the input array 11′ can be pulsed individually, and the output at the detector measured with knowledge of the spatial location and direction of the input beam.

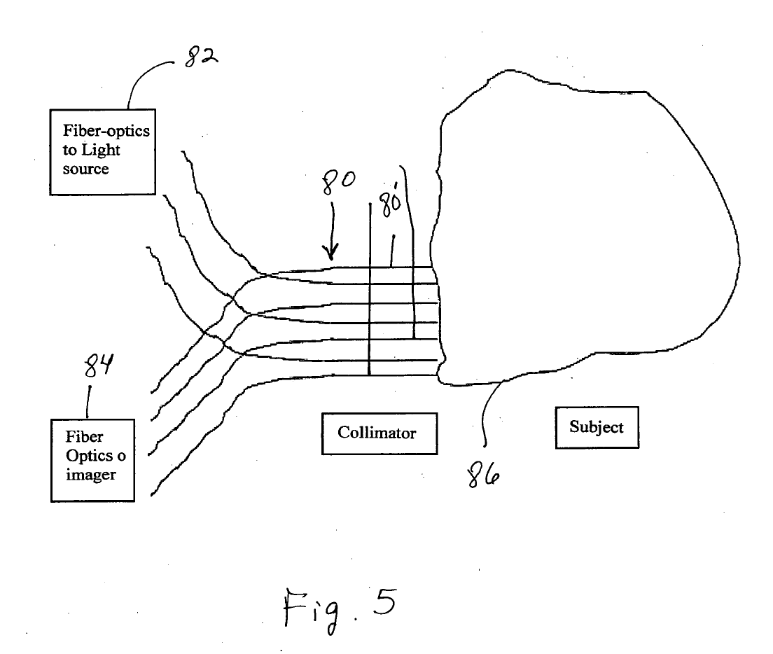

Different individual elements 12A′ for the collimator 18″ could be activated by mechanical or electronic means, for example by butting the input end (left end in FIG. 4) bundle of optical fibers 12A′ that feeds the collimator against some array of diodes (in 12′) or butting the collimator directly against some custom-fabricated diode or light array. Alternatively, a micro mirror array (MMA) (see FIG. 8) could be located in 12′ and used, or mechanically by triggering nano activators, for each tube or element 16″. This scheme of butted fibers, though not as clear as a shadow and scattering transmission system, could be used for a “reflectance only” system, which would have advantages, for instance, for Traumatic Brain Injury assessment (assessment of vascularization in the cerebral cortex), or in other situations or geometries where the transmission system and collimator described above are difficult to use as the brain and skull are more rigid than, for example, the human breast, and therefore not subject to a scattering analysis. In this case, the utility of the detector collimator 80 (see FIG. 5) is to prevent light from the source 82 which is scattered from making it to the detector 84, thereby allowing a substantial increase in signal to noise over a system that accepts photons of all possible trajectories.

In the head-on, or butted-up-against-the-skull design illustrated in FIG. 5, light from the source 82 is fed to the skull through fiber optics bundles, to the single or combined input/detector collimator 80 (so the photons are going straight in). Then, only photons that undergo reflection directly back into the instrument are able to make it back through the input/detector collimator 80 back through the fiber optic bundles to the detector portion 84. The bundles 80 could be held in a semi-flexible mount system that would allow the bundle to follow the contour of the subject or skull 86.

The basic scheme of this “Head-On system” application is illustrated in FIG. 5: This embodiment allows the device to probe for absorption in layers below the skin, without a transmission system, but measures changes in reflection. Light enters the collimator 80 through fiber optics that are uniformly distributed over the subject area, which is pressed tightly against the collimator at its input end and against the skull or target 86 at its other end. Arranged over the subject uniformly are “detection tubes” located in 80′ that take ballistic photons (say photons with only one reflection) back through the collimator, and then feed them to the fiber optics and back to the detector 80. Areas of brain with excess or inadequate blood flows indicative of Traumatic Brain Injury, could thus be distinguished from normal brain tissue.

In another possible application, the input beam could be synchronized with the arterial (or other body structures) pulse (or other body movements/functions, e.g. breathing), in order to better isolate and delineate key vascularized (or other) structures.

The mathematical solution may be, for example, a global least-squares fit to a model of the scattering medium, where the only free parameters are the coefficients of the micro-targets. We believe that this may be the preferred embodiment of the algorithm. A homogeneous set of micro-targets with the expected dominant biological component—say fibrous tissue or fat, for mammary glands—can be the starting point for the calculation, with plausible guesses for differences between the observed and the re-constructed underlying tissue leading to the next steps of the iteration. We assert that in this way we can develop an algorithm that will converge quickly. For example, in the case of a uniformly fatty and homogeneous mammary gland, one could assume that the micro-targets are all fat and have the same S, A, and T, then subtract that assumption from the observed pattern of light. Then, from the residuals in the case of one small volume of, say, cancerous cells—say s, a, and t and its characteristic pattern—a scattering and absorption pattern would be apparent from the residuals. Finally, in the software, one manages to fit the spatial distribution of the targets and the coefficients of the residuals. One could insert into the global solution in the software a small object with the characteristics of a cancer cell into the assumed target and recalculate to find out whether any residuals exist, or make other corrections.

The strength of this method is that a fairly simple model can be imposed on the target and quickly calculated, leading to a difference image which contains more information about the underlying tissues in the patient. (Adding only one simulated cancer cell to the solution will lead to a better fit, even if the cancer distribution (simulated and perhaps actual) is more complex. Hence, this method should converge rapidly yielding an image for the simulated and actual cancer cells. This methodology is likely to have particular application in determining and assuring successful remission of cancerous cells following chemotherapy or surgical resection.

Collimator and Phase-Space Reduction.

Though collimators are used in almost all imaging devices, the innovation that we are claiming is the development of a device that is able to illuminate one tube of the collimator (or multiple tubes with different frequencies or polarizations (if the interference can be discriminated)) at a time, in addition to a unique design that greatly decreases angular dispersion and input position of the input beam, and results in the detection of only a phase-space purified output beam, largely devoid of reflection, scattering, components, etc. Hence, we can more easily understand the scattering and absorption for that element individually, with no moving parts and no confusion from light from other parts of the illuminating source. The idea is to sequence the input beam (fire off one “tube” of the collimator at a time, as needed). One proposed way of doing this is to have a micro mirror array or video computer projector in front of the input collimator. In that way we have one “tube” (which we keep track of) illuminate the breast, brain or other target, then use the collected light to start analyzing the underlying tissues, as above. We then fire off the next one, collect light, and so on.

The Collimator on the detector side or the whole detector/illuminator scheme can have a hole or blank spot for insertion of catheters or making marks on the target, for example.

We can add the data from individual tube firings all up, if we wish, in order to get the easy, first-order shadow image too.

FIG. 6 show a refinement of the proposed embodiment where we have now included the LCD “multiplexer” 12′″ from a video projector as part of the multi-element phase-space reduction input system. Most of the time, the LCD remains opaque, and introduces no light component itself. When a tube 16′″ is desired to be illuminated, the LCD mask opens up just at that exact point in the LCD mask and it becomes transparent.

FIG. 6 is a two-dimensional slice through a multi-element phase-space reduction input system (4-elements). Each fiber or matrixed light source 12′″ of the input array (including light source 12A′″) can be pulsed individually, and its output measured with knowledge of the spatial input of the and the direction of the resultant beam.

The collimator works as follows:

Light that is going straight gets through—light that is going crooked or bounces off the walls of the tube 16 gets stopped.

FIG. 6A list various parameters for constructing the present invention, including dimensions and wave lengths for the light source, collimators, input and output, and detectors.

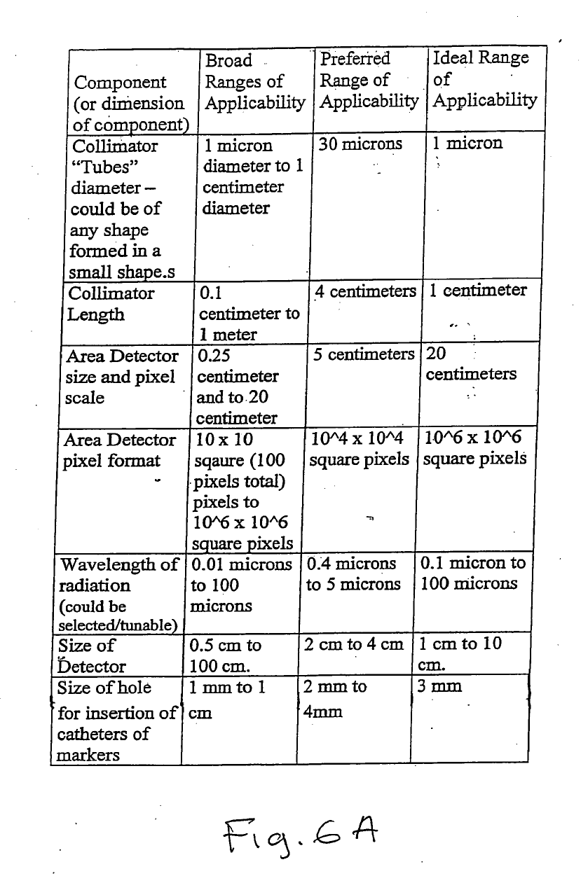

FIG. 7 shows the path (heavy arrows) of photon that is not on axis. Note the blocking stops (baffles 16A or ends 52) in the collimator tube 16 that stop photons that are reflected off of the walls. All interior walls 16A and baffles or walls are black and/or absorbing. This particular geometry mitigates against photons that reflect off of the wall or baffles from making it through the tube.

The collimator element shown in FIG. 3A would work in a similar manner but is easier to construct as there are no interior baffles to form, and the absorbing or black coating can be applied on the exterior, rather than on the interior of the element, the exterior being possible as the glass of section 22 would be transparent, absence the black coating.

Also if desired a mirror array 12E could be interposed between the light source 12 and collimator 18 to control the light into the collimator such as shown in FIG. 8. The remainder of the input unit could be similar to that shown in FIG. 1, with if desired, a filter/polarizer provided.

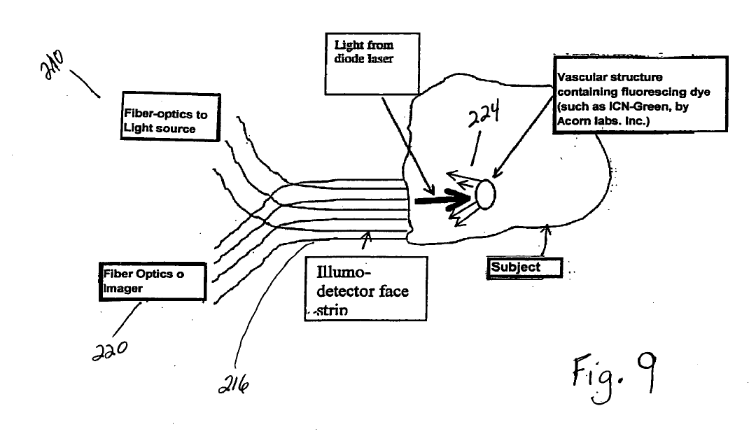

With respect to FIGS. 9 to 14, the same reference numerals (but 200 numbers higher—e.g., 24 in FIG. 1 would be 224 in FIG. 9) are used for the same or similar elements previously described. FIG. 9 illustrates the use of a fluorescing agent such as ICN-Green say for cranial analysis such as in connection with TBI. The collinear light 224, is to some degree reflected, scattered, absorbed and transmitted through the target 226. Such light could excite selectively, fluorescent molecules in the target of interest.

In one aspect, the present invention incorporates or utilizes fluorescing dyes or agents to help acquire the images. Such approach will result also in the presence, after excitation, of emitted or fluoresced light from the target in the subject. In order to collect the scattered, reflected, fluorescing, and transmitted light, if any, a detector portion 236 is provided, and can comprise an output collimator 238 similar to collimator 218, and output filters and/or polarizers 240, similar to filters/polarizers 212 and a detector unit 242. Light from the target enters the output collimator 238, and if used the filters/polarizers 240, and then the detector unit 242.

As the present invention can be used with fluorescing materials or dyes, if a long enough duration or time fluorescing agent would be used, one could pulse the target, and then after the pulse of exciting radiation has subsided, enable the detectors, to substantially detect only fluorescing atoms or structures, and with less background noise from scattered light.

FIG. 9 illustrates in detail, a schematic of one embodiment of the trans-cranial imaging system. This embodiment allows the device to probe for absorption and reflection in layers below the skull, without a transmission system, but measures distribution of dye, which is in the vascular system. Light enters the illumo-detector 210 through fiber optics 212 that is distributed over the subject area 214, which is pressed tightly against the collimator. Arranged over the subject uniformly are “detection tubes” 216 that take ballistic photons (say photons with only one reflection) through the collimator, and then feed them to the fiber optics 218 and back to the detector 220. FIG. 9 shows how the collinear light 224 scatters internally on an object or target containing fluorescing agent inside the human skull.

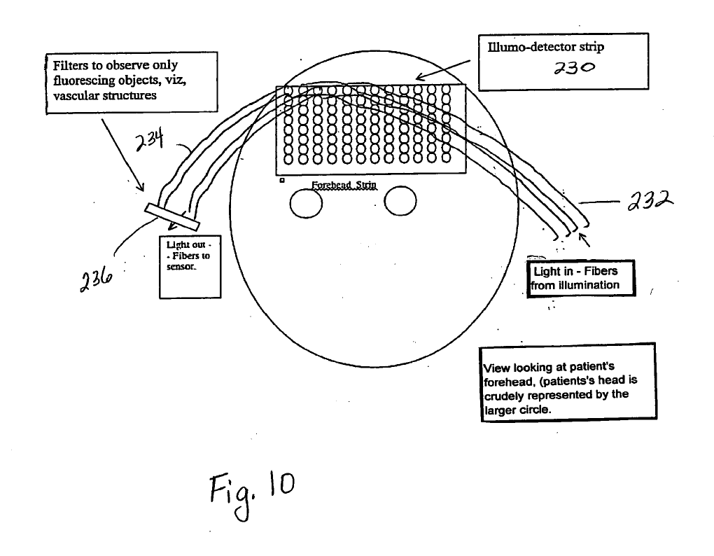

FIG. 10 shows the details of the illumo-detector strip or unit 230, which can be placed on the patient's head. In this embodiment of the apparatus, light of a given polarization is “pumped” into the subject, by pulsing individual fibers 232, which then are injected via the illumo-detector strip 230. The light encounters the fluorescing dye, excites radiation of a longer wavelength, and such light is recovered by the fibers 234 going to the detector 236, on the illumo-detector strip.

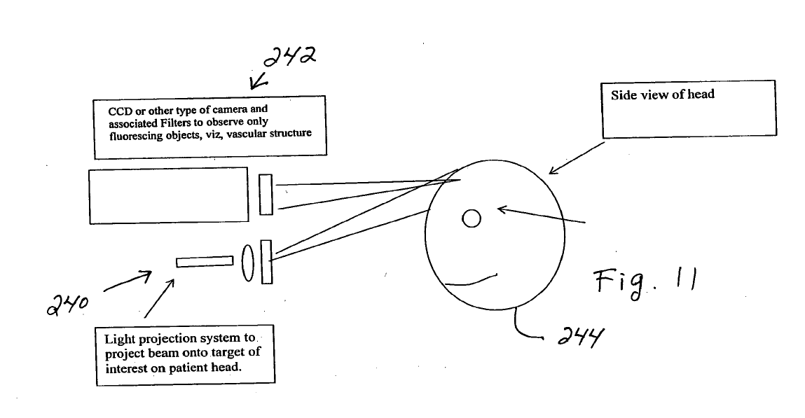

FIG. 11 shows the present invention, including the light source 240, detector 242 the fluorescing agent (in patient 244) without the use of the strip of FIG. 10.

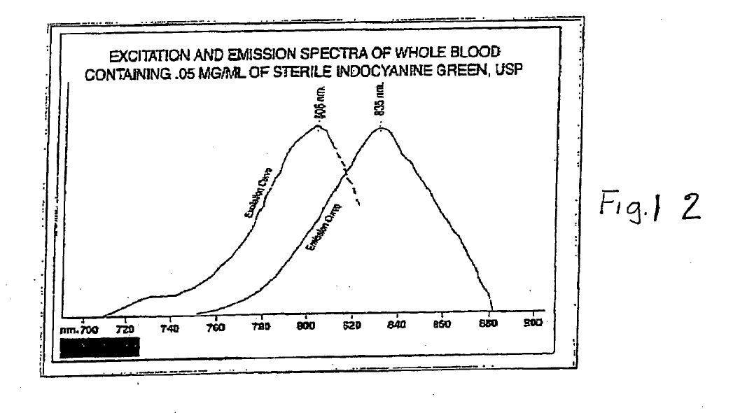

FIG. 12 illustrates emission and emitted radiation of ICN-Green, a typical fluorescing dye. The light from the image without ICN-Green or other fluorescing dye which also could include light that is scattered or emitted from various objects in the beam, can then be subtracted from the image with the fluorescing dye. The result (with dye less without dye or vice versa) defines in greater clarity details of the object of interest. Further, a simpler light source that uniformly or non-uniformly illuminates the target of interest, could excite the fluoresced molecules, and only light from the fluoresced molecules could be images or data acquired from such fluorescing molecules. Data and images taken in the absence of the fluorescing agent could be compared to or subtracted from images that contain the fluorescing agents, so one is left with only light signals from structures of interest or the area immediately around such structures of interest.

When dealing with fluorescing material point objects may be characterized by the coefficients of S, A, F and T, and s, a, f and t, respectively, situated next to each other (wherein S, A, T are as defined above and F and f are the fluorescing coefficient. If they are illuminated, and S, A, T and s, a, t, f of each material are known (this assumes the smaller object is the only one fluorescing), if in addition we are able to place some constraints on the geometry, then it is possible to solve for the underlying spatial distribution of the tissue in question. A target that emits fluorescing light will allow greater depth and spatial resolution, as its light is emitted at a wavelength of higher transmission through the overlying material, and also the signal from such an object does not have any contribution of light from the incoming beam, allowing greater fidelity in image reconstruction.

A slightly different case involves one point objects one of which shines by emitting fluorescent light. The point object's light scatters out of the target's body, eventually into the detector. By solving for scattering and transmission along the path from the object to the detector, one can significantly reduce the errors in position of the fluorescing object. This system has the advantage of not being sensitive to light from the input of the illumo-detector, since this light is at a different wavelength than is detected, with our envisioned filters.

Other wavelengths of interest, for example micro-waves might be used to excite the fluorescing media or agent.

In another possible embodiment, the target could include other materials, which have been made with small amounts of fluorescing materials, either on purpose, or added to the materials during manufacturing or for testing. For example, light weight composite materials would show defects, such as broken fibers or other structural problems, deep in the materials. The same methods used for studying targets and surrounding areas in humans could be applied to these materials, and greatly increase the testing fidelity before, during, or after assembly into its final structure.

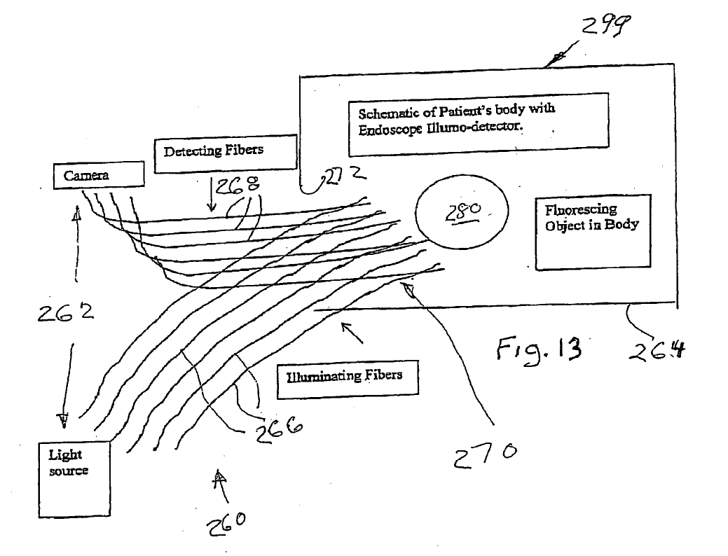

FIG. 13 shows the present invention can be extended to endoscopic systems 260 and can be used with or without a fluorescing agent. Another use of this system is to provide an illumo-detector element 262 of a geometry designed to be placed inside a patient 264 by the well-known methods of endoscopy (see, e.g., http://en.wikipedia.org/wiki/Endoscopy). In this case, the illuminating fibers 266 are bundled together with the detector fibers 268 in an endoscopic probe 270 that can be inserted into a suitable incision, space, cavity 272 or orifice in the patient 264. Then, the illuminating fibers 266 could pulse, either individually, in groups, or simultaneously and enable the object of interest 280 that may or may not contain fluorescing dyes to be illuminated. Then, either individually or in groups, the light or fluorescently emitting light signal could be received by the detector fiber optics 268, and then either individually, in combination of fibers, or all simultaneously could form an image of the object of interest. As previously noted the detector may have a collimator provision at its distal end and photon entry place, or the collimator may be located in the fiber optics spaced away from the distal end, or even dispensed with. The latter two constructions permit a more compact endoscopic probe, with the collimator located downstream from the distal end and/or placed external of the patient and/or dispensed with. With the collimator downstream many of the non centered photons (those reflected off of the outer surface of the fiber optics) would still be trapped by a downstream collimator. Likewise, while the collimator for the illuminator is preferably at or near its discharge end, it could be placed anywhere between the light source and the end.

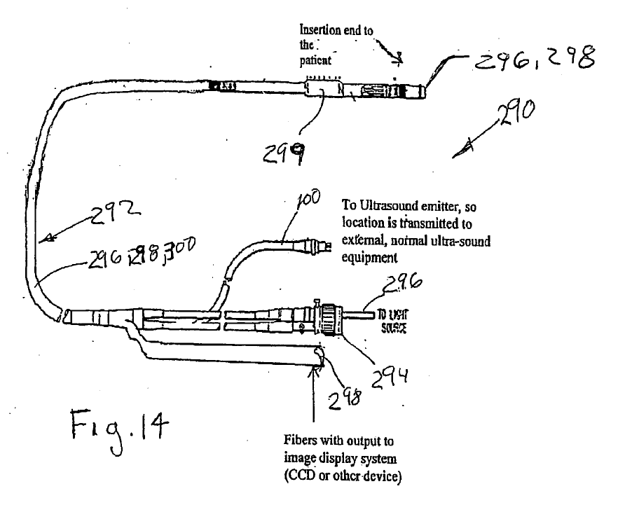

FIG. 14 shows an endoscope 290 for such an application. The endoscope 290 has distal end 288 of a coil 292, of illuminating and detecting fiber optics therein which includes a fish-eye wide field of view optic 294 (including fiber optics 300 for the same) enabling a field of view of about 270 degrees to enable viewing where the endoscope is looking and also its location. Illuminating fiber optic are at 296 with small lenses for dispensing light over the field of view of interest. Detecting fiber optics 298 are also co-parallel with fiber optics 296. In the alternative with suitable switching a single set of optic fibers could be used for all three functions (illuminating, viewing and detecting). Absolute location of the endoscope can be by ultrasonic transducer 299 such as disclosed in the Silverstein et al U.S. Pat. No. 4,462,408, which is hereby incorporated by reference.

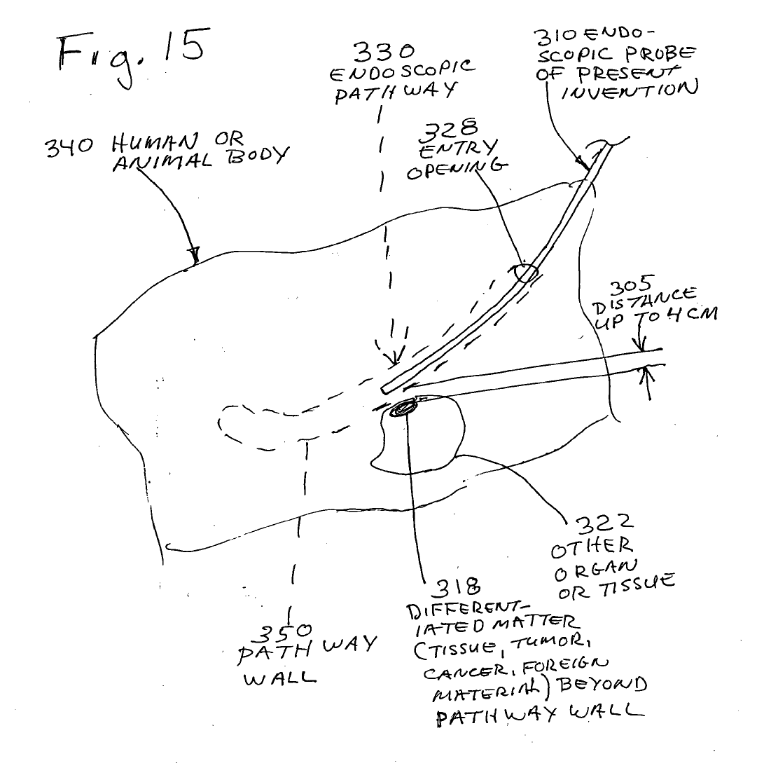

Referring to FIG. 15, the present invention, with or without fluorescence, can form images of at considerable depth (from above, or on the surface to at least 1 cm and even to 4 cm in and beyond the surface) in tissue bone, organs, and/or through endoscopic forms of probes 310. The present invention may detect tumors, cancer or other differentiated tissue or matter (say swallowed objects) 318 in tissue or organs 322 surrounding the endoscopic pathway 330 (say colon intestine, esophagi, bronchial tube, etc.) used to traverse the endoscopic probe. Thus, the human or animal body 340 can be more extensively explored not only to detect differentiated tissue (tumors, cancers, etc.) 318 using naturally formed openings or spaces to insert the endoscopic probe 310 and detect matters 318 in adjacent structures, tissue or organs 322, actually hidden visually by the wall 350 of the pathway 330. Thus, with the present invention in endoscopic form one can detect anomalies or differentiated matter (tumor, cancer, etc.) above, on, in and beyond the endoscopic pathway wall.

These endoscopic probes and methods of the present invention could be used to explore the surfaces and depths below the surface of esophagus, colon, bronchial tube and/or in any known or to be known endoscopic applications. The present invention using such endoscopic probes to provide penetration and information on tissues and structures say of 1 cm to 4 cm into and below the surface. Such probes suitably built could also have industrial applications. Likewise, the endoscopic applications could be used with or without fluorescing dyes and materials.

The power consumption for the light source and particularly the power input into the patient or material being investigated is low and less than one kilowatt, and more likely between 10 to 200 watts with about 30 watts or less being preferred. This is advantageous as no special circuits are needed to power the device. A greater advantage is that the power input on a human or animal is such that there is no danger of burns, except when the collimated light (ultraviolet, visible, or infrared) is concentrated by targeting say a tumor.

While the preferred embodiments of apparatus and steps of the method for practicing the present invention have been disclosed and described, it should be understood that variations thereof and equivalent elements and steps fall within the scope of the invention described in the appended claims.

1. A method for detecting a body in an object which body is below the surface of the object, comprising:
illuminating the body and object with light,
detecting two or more of the light reflected by, scattered by, and
transmitted by the body
and object,
analyzing the detected light for purposes of forming an image of the body in and below the surface of the object,
whereby the different characteristics of two or more of scattering, reflecting and transmitting of light by the body and object permits forming the images of the body in and below the surface of the object.

2-3. (canceled)

4. The method of claim 1, comprising the step of using illuminating light of different frequencies.

5. The method of claim 1, comprising the step of polarizing the light before and/or after the illuminating step.

6. The method of claim 1, comprising the step of using the light to heat the body in and below the surface of the object to a temperature sufficient to affect the body without damaging the object.

7. The method of claim 1, wherein said body is a tumor and comprising the step of heating the tumor with the light to a temperature of at least 113° F. sufficient to kill the tumor.

8. The method of claim 1, wherein said body is at least 1 cm in depth below the body's surface.

9. A method of claim 8, wherein said body is up to 4 cm in depth below the body's surface.

10. The method of claim 1, wherein said body is at least 1 cm and up to 4 cm below the body's surface, comprising the steps of:
using illuminating light of different frequencies, and
polarizing the light before and/or after the illuminating step.

11. The method of claim 1, wherein said body is at least 1 cm below the body's surface, comprising the step of using the light to heat the body to a temperature sufficient to effect the body without damaging the object, wherein said body is a tumor and comprising the step of heating the tumor with the light to a temperature of at least 113° F. sufficient to kill the tumor, while the area around the tumor remains at a lower temperature.

12. An apparatus for detecting a body in an object which body is below the surface of the object, comprising a light source for illuminating said body below the surface of the object,
detection means for detecting two or more of the light scattered by, reflected by and transmitted through said body and object to form an image of said body in and below the surface of said object.

13-18. (canceled)

19. An apparatus as in claim 12, wherein the illuminating light is one or more of altered in frequency, filtered, and/or polarized.

20. An apparatus as in claim 12, wherein the detected light is one or more of altered in frequency, filtered, and/or polarized.

21. An apparatus as in claim 12, wherein said apparatus is capable of heating the body in the object to alter the body without damaging the object.

22. An apparatus as in claim 21, wherein said body is a tumor and said apparatus heats said tumor to a temperature sufficient to kill said tumor.

23-26. (canceled)

27. An apparatus as in claim 12, wherein said body is at least 1 cm in depth below the body's surface, wherein said apparatus is capable of heating the body in the object without damaging the object, said body being a tumor and said apparatus heating said tumor to a temperature of at least 113° F. sufficient to kill said tumor.

28-53. (canceled)

54. A method for detecting a body in an object and below the surface of the object, comprising:
illuminating the body and object with light,
causing one of the body and object to at least one of emit and fluoresce,
detecting the light reflected by, scattered by, absorbed by and then at least one of emitted fluoresced by one or more of the body and object,
analyzing the detected light for purposes of forming an image of the body in and below the surface of the object, whereby the different characteristics of scattering, reflecting, at least one of emitting and fluorescing light by the body and object permits forming the image of the body in the object.

55-56. (canceled)

57. The method of claim 54, comprising the step of using illuminating light of different frequencies.

58. The method of claim 54, comprising the step of polarizing the light before and/or after the illuminating step.

59. The method of claim 54, comprising the step of using the light to heat the body to a temperature sufficient to effect the body without damaging the object.

60. The method of claim 54, wherein said body is a tumor and comprising the step of heating the tumor with the light sufficient to kill the tumor.

61. The method of claim 54, comprising the steps of spacially defining the input beam of the light scattered by fluoresced by, reflected by, and/or transmitted through the object and/or body, using illuminating light of different frequencies, polarizing the light before and/or after the illuminating step.

62. An apparatus for detecting a body in and below the surface of an object, comprising a light source, means for fluorescing one of said body and object, detection means for detecting the light scattered, reflected, fluoresced and/or transmitted to form an image of said body in and below the surface of said object.

63-68. (canceled)

69. An apparatus as in claim 62, wherein the illuminating light is one or more of altered in frequency, filtered, and/or polarized.

70. An apparatus as in claim 62, wherein the detected light is one or more of altered in frequency, filtered and/or polarized.

71. An apparatus as in claim 62, wherein said apparatus is capable of heating the body in the object to alter the body without damaging the object.

72. An apparatus as in claim 71, wherein said body is a tumor and said apparatus heats said tumor to a temperature sufficient to kill said tumor.

73-75. (canceled)

76. An apparatus as in claim 62, further including said collimator means comprises one or more fiber optic section, said section having an absorbing surface on its outer surface and end surfaces and at least two small openings in said end surfaces to permit transmission of light.

77-83. (canceled)

84. A method as in claim 1, wherein said illuminating taking place in an endoscope.

85. A method as in claim 84, wherein said detecting the light takes place in an endoscope.

86. A method as in claim 1, wherein said detecting the light takes place in an endoscope.

87. A method as in claim 1, wherein said illuminating and said detecting and can detect and form an image to a depth of at least 1 cm in and below the surface of the object.

88. A method as in claim 84, wherein said illuminating and said detecting is to a depth of up to about 4 cm in and below the surface of the object.

89. A method as in claim 84, comprising the step of moving the endoscope in an endoscopic pathway.

90. A method as in claim 89, comprising the step of illuminating and detecting at least three of: above, on, in and beyond the pathway.

91. A method as in claim 84, wherein said pathway has a pathway wall and said illuminating and detecting occurs beyond the pathway wall.

92. A method as in claim 91, wherein said illuminating and detecting occurs from 1 cm to 4 cm beyond the pathway wall.

93. An endoscope for use in an animal or human tissue comprising an illuminator and a detector, said illuminator illuminating and said detector detecting to a depth of at least one centimeter below the surface of the tissue.

94. An endoscope as in claim 93, said illuminator illuminating and said detector detecting to a depth up to 4 centimeters.

95. An endoscope as in claim 93, further including a collimator for said detector which is located at the distal end of said detector.

96. An endoscope as in claim 93, further comprising a collimator of said detector which is located a distance downstream from the distal end.

97. An endoscope as in claim 93, including means for locating the endoscope.

98. A method as in claim 1, wherein said illuminating taking place in an endoscope, said detecting the light takes place in an endoscope, and said illuminating and said detecting illuminate and can detect and form an image to a depth of at least 1 cm in the object.

99. A method as in claim 98, wherein said illuminating and said detecting is to a depth of up to about 4 cm.

100. A method as in claim 98, comprising the step of moving the endoscope in an endoscopic pathway.

101. A method as in claim 100, comprising the step of illuminating and detecting at least three of above, on, in and beyond the pathway.

102. A method as in claim 100, wherein said pathway has a pathway wall and said illuminating and detecting occurs beyond the pathway wall.

103. A method as in claim 100, wherein said illuminating and detecting occurs from the inner surface of the pathway wall to 4 cm beyond the pathway wall.

104. A method as in claim 1, wherein said illuminating comprises the step of illuminating a plurality of light sources.

105. A method as in claim 104, comprises the step of illuminating the plurality of light sources sequentially.

106. A method as in claim 1, wherein said detecting step comprise the detecting the light in a plurality of detectors.

107. A method as in claim 106, wherein said detecting step comprised detecting in the plurality of detectors sequentially.

108. A method as in claim 106, wherein said illuminating comprises the step of illuminating said plurality of light sources sequentially.

109-111. (canceled)

112. The method of claim 1, comprising the step of powering the illuminating with one kilowatt or less.

113. The method of claim 114, wherein the powering step comprises providing between 10 to 200 watts power.

114. The apparatus as in claim 12, having a light source of less than 1 kilowatt.

115. An apparatus as in claim 114, wherein said light source is from 10 to 200 watts.

116. A method as in claim 1, wherein said illuminating is providing one of ultraviolet, visible, or infrared light

117. An apparatus as in claim 12, wherein said light source is one of ultraviolet, visible or infrared light.

118. A method for detecting traumatic brain injury and its decreased blood flow in a living human head, including any hair, scalp, skull bone present and brain tissue and its blood vessels therein, comprising the steps of:
illuminating the human head with light,
detecting the light reflected by and/or scattered by the human head, including any hair, scalp, skull bone present, and brain tissue and its blood vessels therein,
analyzing the detected light for purposes of forming an image through any hair, scalp and skull bone present of the brain tissue and blood vessels therein, and
forming an image of said brain tissue and blood vessel's therein indicating traumatic brain injury,
whereby the different characteristics of scattering, reflecting and transmitting of light by any hair, scalp, skull bone present, and brain tissue and its blood vessels therein form an image of traumatic brain injury in the brain tissue.

119. A method as in claim 118, comprising the further step of injecting a fluorescent into the human brain blood vessels, and carrying out the steps of claim 118 to form an image of the brain and its blood vessels.

120. A method as in claim 119, comprising carrying out the steps of claim 118, creating a first image, and carrying out the steps of claim 119 creating a second image and then subtracting the first image from the second image.

121. The method of claim 119, wherein a CCD camera is used to detect said light.

122. The method of claim 119, wherein ICN green is the injected fluorescent.

123. The method of claim 118, wherein the step of detecting includes distinguishing small signals from background noise.

124. The method of claim 123, wherein the step of detecting is using a camera.

125. The method of claim 124, wherein the step of using a camera comprising using a CCD camera.

126. The method of claim 118, wherein the steps of illuminating comprising using two or more light sources.

127. The method of claim 126, wherein the steps of illuminating comprising the step of sequencing said two or more light sources.

128. The method of claim 118, wherein the step of detecting comprising using two or more means for detecting.

129. The method of claim 128, wherein the step of detecting comprises the step of sequencing said two or more means for detecting.

130. The method of claim 118, comprising transmitting the illumination at one frequency and detecting the light reflected by and/or scattered by at another frequency.

131. An apparatus as in claim 12, wherein said light source has a power level of approximately 1 watt spread over a few square centimeters.

132. A method as in claim 1, wherein said illuminating is at a power level of 1 watt spread over a few square centimeters.
